(19) United States
(12) Patent Application Publication
(10) Pub. No.: US 2026/0198889 A1
(43) Pub. Date: Jul. 16, 2026

(54) ULTRASOUND IMAGING METHOD, ULTRASOUND IMAGING DEVICE, AND NON-TRANSITORY COMPUTER-READABLE MEDIUM

(71) Applicant: GE Precision Healthcare LLC, Waukesha, WI (US)

(72) Inventors: Zhiwen Wang, Wuxi (CN); Gang Liu, Wuxi (CN); Yuanlong Meng, Wuxi (CN); Yaning Li, Wuxi (CN)

(21) Appl. No.: 19/450,463

(22) Filed: Jan. 15, 2026

(30) Foreign Application Priority Data
Jan. 16, 2025 (CN) 202510072100.3

(51) Int. Cl. A61B 8/08; A61B 8/00

(52) U.S. Cl. CPC A61B 8/0866; A61B 8/461; A61B 8/48; A61B 8/5223; A61B 2503/02

(57) ABSTRACT

An ultrasound imaging method including collecting volumetric ultrasound data by using an ultrasound probe; setting a target plane according to the volumetric ultrasound data; and indicating that movement of the ultrasound probe is to be performed according to position information of the target plane, so that the ultrasound probe is capable of scanning the target plane, wherein the target plane is a plane of a fetal spine that satisfies a set condition. An indication to move the ultrasound probe can be provided to a physician, thereby reducing the workload of the physician.

### CROSS-REFERENCE TO RELATED APPLICATION

This application claim priority to Chinese Patent Application No. 202510072100.3, which was file on Jan. 16, 2025 at the Chinese Patent Office. The entire contents of the above-listed application are incorporated by reference herein in their entirety.

### TECHNICAL FIELD

The present specification relates to the technical field of medical imaging and, in particular, to an ultrasound imaging method, an ultrasound imaging device, and a non-transitory computer-readable medium.

### BACKGROUND

Ultrasound imaging technology can use an ultrasound probe to scan a site of interest to obtain ultrasound data of the site of interest. By processing the ultrasound data, an ultrasound image of the site of interest can be generated. Ultrasound imaging technology is generally considered to be safer than ray type imaging technology. Therefore, ultrasound imaging technology is more suitable for some special use scenarios, for example, examination and imaging of a fetus.

It should be noted that the above introduction of the background is only for the convenience of clearly and completely describing the technical solutions of the present application, and for the convenience of understanding for those skilled in the art.

### SUMMARY OF THE INVENTION

When performing an ultrasound examination on a fetus, a physician can operate an ultrasound probe to scan a site of interest in the fetus to obtain an ultrasound image of the site of interest. The fetal spine is a conventional site of interest. In the related art, the physician needs to autonomously operate the ultrasound probe to scan the fetal spine. The inventor has found that the related art above has certain limitations. For example, unlike a conventional region of interest, the position of the fetus in the body of the mother is not fixed, which results in uncertainty in the position of the fetal spine in the body of the mother. In addition, the spine itself may also bend to different angles, which makes the prediction of the spine position more difficult. The physician needs to spend a long time moving the ultrasound probe to scan the site of interest. This increases the workload of the physician.

In view of the above technical problems or other similar problems, the embodiments of the present specification provide an ultrasound imaging method, an ultrasound imaging device, and a non-transitory computer-readable medium, to indicate to a physician to move an ultrasound probe, thereby reducing the workload of the physician.

The embodiments of the present specification provide an ultrasound imaging method, including:
collecting volumetric ultrasound data by using an ultrasound probe;
setting a target plane according to the volumetric ultrasound data; and
indicating that movement of the ultrasound probe is to be performed according to position information of the target plane, so that the ultrasound probe is capable of scanning the target plane, wherein the target plane is a plane of a fetal spine that satisfies a set condition.

The embodiments of the present specification further provide an ultrasound imaging device, including:
an ultrasound probe, configured to collect volumetric ultrasound data; and
a processor, configured to: set a target plane according to the volumetric ultrasound data; and indicate that movement of the ultrasound probe is to be performed according to position information of the target plane, so that the ultrasound probe is capable of scanning the target plane, wherein the target plane is a plane of a fetal spine that satisfies a set condition.

The embodiments of the present specification further provide a non-transitory computer-readable storage medium for storing a computer program, which when executed by a computer, enables the computer to execute the above ultrasound imaging method.

In the technical solutions of the embodiments of the present specification, an ultrasound probe may be used to collect volumetric ultrasound data. The target plane can be set in the volumetric ultrasound data. An indication to move the ultrasound probe can be provided according to position information of the target plane, so that the ultrasound probe is capable of scanning the target plane. Thus, by indicating to the physician to move the ultrasound probe, the physician can operate the ultrasound probe targetingly. This simplifies operations for the physician, shortens the time required for the physician to operate the ultrasound probe, and reduces the workload of the physician. In addition, the target plane is the plane of the fetal spine that satisfies the set condition. The ultrasound probe can scan the target plane of the fetal spine to obtain a two-dimensional ultrasound image of the target plane, which is convenient for the physician to diagnose the fetal spine.

With reference to the following description and drawings, implementations of the embodiments of the present specification are disclosed in detail, and the manners in which the principles of the embodiments of the present specification can be employed are illustrated. It should be understood that the implementations of the present specification are not limited in scope thereby. Within the spirit and the scope of clauses of the appended claims, the implementations of the present specification comprise many changes, modifications, and equivalents.

### BRIEF DESCRIPTION OF THE DRAWINGS

The included drawings are used to provide further understanding of the embodiments of the present specification. The drawings constitute a part of the specification, and are used to illustrate the implementations of the present specification, and together with the written description, explain the technical principles of the embodiments of the present specification. Obviously, the drawings in the following description are only some embodiments of the present specification, and those of ordinary skill in the art may obtain other implementations according to the drawings without the exercise of inventive effort. In the drawings:

FIG. 1 is a schematic architecture diagram of an ultrasound imaging device in some embodiments of the present specification;

FIG. 2 is a schematic diagram of a mechanical scanning ultrasound probe in some embodiments of the present specification;

FIG. 3 is a schematic diagram of a scanning angle range in some embodiments of the present specification;

FIG. 4 is a schematic flowchart of an ultrasound imaging method in some embodiments of the present specification;

FIG. 5 is a schematic diagram of volumetric ultrasound data in some embodiments of the present specification;

FIG. 6 is a schematic diagram of a process of setting a target plane in some embodiments of the present specification;

FIG. 7 is a schematic diagram of a process of rotating an ultrasound probe in some embodiments of the present specification;

FIG. 8 is a schematic diagram of a process of translating an ultrasound probe in some embodiments of the present specification;

FIG. 9 is a schematic diagram of a process of translating an ultrasound probe in some embodiments of the present specification;

FIG. 10 is a schematic diagram of adjusting a scanning angle range in some embodiments of the present specification;

FIG. 11 is a schematic diagram of an interaction interface in some embodiments of the present specification;

FIG. 12 is a schematic diagram of an interaction interface in some embodiments of the present specification;

FIG. 13 is a schematic diagram of an interaction interface in some embodiments of the present specification;

FIG. 14 is a schematic diagram of an interaction interface in some embodiments of the present specification;

FIG. 15 is a schematic diagram of an interaction interface in some embodiments of the present specification;

FIG. 16 is a schematic structural diagram of an ultrasound imaging apparatus in some embodiments of the present specification;

FIG. 17 is a schematic structural diagram of an ultrasound imaging apparatus in some embodiments of the present specification;

FIG. 18 is a schematic diagram of an ultrasound probe in some embodiments of the present specification; and

FIG. 19 is a schematic diagram of a fetal spine in some embodiments of the present specification.

### DETAILED DESCRIPTION

The technical solutions in the embodiments of the present specification will be clearly and completely described below with reference to the drawings in the embodiments of the present specification. Obviously, the described embodiments are only some, but not all, of the embodiments of the present specification. The specific embodiments described herein are merely configured to explain the present disclosure, rather than to limit the present disclosure. All other embodiments obtained by those of ordinary skill in the art based on the described embodiments of the present disclosure fall within the scope of protection of the present disclosure. In addition, relational terms such as “first” and “second” are used merely to distinguish one entity or operation from another entity or operation, and do not necessarily require or imply that any such actual relationship or sequence exists between these entities or operations.

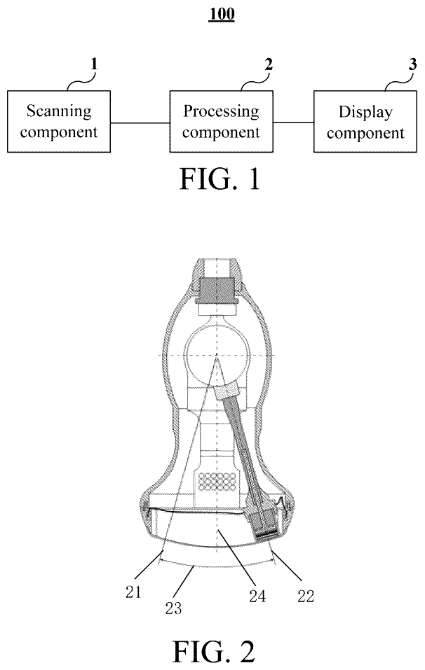

FIG. 1 is a schematic architecture diagram of an ultrasound imaging device in some embodiments of the present specification. As shown in FIG. 1, the ultrasound imaging device 100 may include an ultrasound probe 2 and a processing component 2. The ultrasound probe 1 is configured to emit ultrasound waves and receive echoes of the ultrasound waves, so as to obtain ultrasound data. The processing component 2 may include a processor. The processor may include any one or a combination of: a central processing unit (CPU), a graphics processing unit (GPU), a micro processing unit (MCU), a programmable logic device (FPGA), etc. The processing component 2 can control the ultrasound probe 1 to collect volumetric ultrasound data, can set a target plane according to the volumetric ultrasound data, and can indicate that movement of the ultrasound probe 1 is to be performed according to position information of the target plane, so that the ultrasound probe 1 is capable of scanning the target plane. The target plane is a plane (e.g., a section) of a site of interest (e.g., the spine) of a fetus.

Thus, by indicating to a physician to move the ultrasound probe 1, the ultrasound imaging device 100 enables the physician to operate the ultrasound probe 1 targetingly. This simplifies operations for the physician, shortens the time required for the physician to operate the ultrasound probe 1, and reduces the workload of the physician. In addition, the target plane is a plane of a site of interest of a fetus. The ultrasound probe can scan the target plane to obtain a two-dimensional ultrasound image of the target plane of the site of interest, which is convenient for the physician to diagnose the site of interest.

In some embodiments, working modes of the ultrasound imaging device 100 may include a volumetric scan mode and a two-dimensional scan mode. In the volumetric scan mode, the processing component 2 can control the ultrasound probe 1 to collect volumetric ultrasound data. In the two-dimensional scan mode, the processing component 2 can control the ultrasound probe 1 to collect two-dimensional ultrasound data.

In some embodiments, the ultrasound probe 1 includes an ultrasound probe capable of three-dimensional scanning.

For example, the ultrasound probe 1 is a three-dimensional (3D) ultrasound probe. The three-dimensional ultrasound probe can perform static three-dimensional scanning on the site of interest to obtain volumetric ultrasound data of the site of interest.

For another example, the ultrasound probe 1 is a four-dimensional (4D) ultrasound probe. The four-dimensional ultrasound probe can perform continuous three-dimensional scanning on the site of interest in the time dimension to obtain time-varying volumetric ultrasound data of the site of interest.

In some embodiments, the ultrasound probe 1 may be a mechanical scanning ultrasound probe or an electronic scanning ultrasound probe.

The mechanical scanning ultrasound probe includes a movable transducer element. The transducer element is configured to emit ultrasound waves and receive echoes of the ultrasound waves. The processing component 2 can control the transducer element to move (e.g., oscillate). Thus, volumetric ultrasound data can be collected. Certainly, the processing component 2 can also control the transducer element to stop moving. Thus, two-dimensional ultrasound data can be collected.

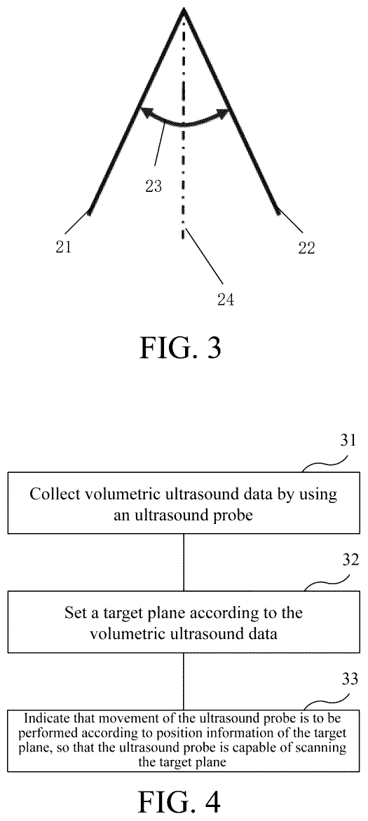

A movement range of the transducer element forms a scanning angle range of the ultrasound probe. For example, FIG. 2 shows a mechanical scanning ultrasound probe. FIG. 3 shows a scanning angle range of a mechanical scanning ultrasound probe. Please refer to FIG. 2 and FIG. 3 together, the transducer element can move between a first position 21 and a second position 22. The first position 21 and the second position 22 form the scanning angle range 23 of the ultrasound probe. A starting position 24 is an intermediate position of the movement range.

The electronic scanning ultrasound probe includes transducer elements arranged in an array. The transducer element is configured to emit ultrasound waves and receive echoes of the ultrasound waves. The processing component 2 can control an activation sequence of the transducer elements to collect volumetric ultrasound data.

Optionally, the ultrasound probe 1 is a mechanical scanning four-dimensional ultrasound probe or a mechanical scanning three-dimensional ultrasound probe.

In some embodiments, the target plane satisfies a set condition. The set condition is used to make the target plane include rich information of the site of interest. The target plane may be a preferred plane (e.g., a section) of the site of interest.

The site of interest has one or more target objects. The target object is a structure or tissue of the site of interest. The set condition may include a quantity condition. The set condition may include: the target plane containing a quantity of target objects greater than or equal to a set quantity. For example, the set quantity may be 5, 7, 8, or the like. In this way, the target plane may contain a greater quantity of target objects. Certainly, the set condition may further include other conditions. For example, the set condition may further include a distribution condition. The set condition may further include: the target objects in the quantity greater than or equal to the set quantity being distributed along an axis of the site of interest in the target plane. In this way, the target plane may contain the axis of the site of interest. Accordingly, the target plane can contain as much information of the site of interest as possible.

That the target objects are distributed along the axis of the site of interest means that the target objects present a distribution trend along the axis. There is no limitation here that the target object must be located on the axis. The target object may be located on the axis or may be located in a region near the axis.

As an example, the site of interest is the spine. The target object may include at least one of a conus medullaris (cm) and a vertebral body (vb). For example, the target plane contains a conus medullaris and vertebral bodies in a quantity not less than N. N may be 5, 7, 8, or the like. A line connecting a plurality of vertebral bodies in the target plane can constitute an axis of the spine. The connecting line may be a line connecting any positions on the plurality of vertebral bodies. Alternatively, the connecting line may be a line connecting the centers of the plurality of vertebral bodies.

In some embodiments, the physician can move the ultrasound probe 1. A pose of the ultrasound probe 1 can be adjusted through movement, so that the ultrasound probe 1 is capable of scanning the target plane. The ultrasound probe 1 can scan a plurality of scanning planes. The processing component 2 can determine that the ultrasound probe 1 can scan the target plane when the target plane is any one of the plurality of scanning planes. Alternatively, the processing component 2 can determine that the ultrasound probe 1 can scan the target plane when the target plane is a specific scanning plane among the plurality of scanning planes. The specific scanning plane is a plane where a scanning axis of the ultrasound probe 1 is located. A direction of the scanning axis is a vertical direction. Reference is made to FIG. 2. The specific scanning plane is a scanning plane where the transducer element is at the starting position 23. The ultrasound probe 1 has better scanning quality for the specific scanning plane. Thus, determining that the ultrasound probe 1 can scan the target plane when the target plane is the specific scanning plane can improve the quality of the two-dimensional ultrasound image of the target plane.

In some embodiments, the ultrasound imaging device 100 may further include a display component 3. The display component 3 may be a display.

In some embodiments, the processing component 2 can perform processing (e.g., rendering) on the volumetric ultrasound data to obtain a volumetric ultrasound image. The display component 3 can display the volumetric ultrasound image. The volumetric ultrasound image is a three-dimensional ultrasound image or a four-dimensional ultrasound image.

In some embodiments, the processing component 2 can obtain first marking information. The first marking information is used to indicate a movement direction of the ultrasound probe 1. The display component 3 can display the first marking information. Thus, the physician can be visually guided to move the ultrasound probe 1. The physician can move the ultrasound probe 1 according to the movement direction indicated by the first marking information.

In some embodiments, during movement of the ultrasound probe 1 by the physician, the processing component 2 can obtain a two-dimensional ultrasound image of a current scanning plane. The display component 3 can display the two-dimensional ultrasound image of the current scanning plane. Thus, during movement of the ultrasound probe 1 by the physician, displaying the two-dimensional ultrasound image of the current scanning plane in real time makes it easy for the physician to intuitively know the current scanning plane of the ultrasound probe 1. The ultrasound probe 1 can scan a plurality of scanning planes. The above current scanning plane may be any one of the plurality of scanning planes, or may be a specific scanning plane among the plurality of scanning planes.

In some embodiments, the processing component 2 can obtain second marking information. The second marking information is used to indicate that the movement of the ultrasound probe 1 is to be stopped. The display component 3 can display the second marking information. Thus, the physician can be guided visually to stop moving the ultrasound probe 1. The physician can stop moving the ultrasound probe 1 according to the indication of the second marking information.

In some embodiments, the processing component 2 can control the ultrasound probe 1 to collect two-dimensional ultrasound data of the target plane, and can perform processing (e.g., rendering) on the two-dimensional ultrasound data of the target plane to obtain a two-dimensional ultrasound image of the target plane. The display component 3 can display the two-dimensional ultrasound image of the target plane. The two-dimensional ultrasound image of the target plane is used for diagnosis by the physician.

In addition, the volumetric ultrasound data can be obtained by synthesizing (e.g., interpolating) two-dimensional ultrasound data of the plurality of scanning planes. For example, the ultrasound probe 1 can collect the two-dimensional ultrasound data of the plurality of scanning planes. The processing component 2 can synthesize (e.g., interpolate) the two-dimensional ultrasound data of the plurality of scanning planes to obtain the volumetric ultrasound data. In the related art, the two-dimensional ultrasound data can be extracted from the volumetric ultrasound data, and the two-dimensional ultrasound image can be generated according to the extracted two-dimensional ultrasound data. The volumetric ultrasound data is obtained by synthesis. The two-dimensional ultrasound image generated according to the extracted two-dimensional ultrasound data has poor quality. In this embodiment, the target plane can be directly scanned, and the two-dimensional ultrasound image can be generated according to the scanned two-dimensional ultrasound data. Thus, the two-dimensional ultrasound image generated in this embodiment has higher image quality (e.g., resolution).

A specific scenario example according to an embodiment of the present specification will be described below. The scenario example is only for better understanding the technical solutions according to the embodiments of the present specification, but does not constitute inappropriate limitations on the technical solutions according to the embodiments of the present specification.

Ultrasound diagnosis of the fetal spine is an important prenatal examination. In the related art, the physician autonomously operates the ultrasound probe to scan the fetal spine, so as to obtain the ultrasound image for diagnosis. The inventor has found that the related art above has certain limitations. For example, the position of the fetus in the body of the mother is not fixed but may change continuously, which results in the uncertainty of the position of the fetal spine in the body of the mother. In addition, the spine itself can also be bent toward different angles, which makes the prediction of the spine position more difficult. The above factors make it take a long time for the physician to operate the ultrasound probe, which increases the workload of the physician and thus extends the time of prenatal examination. For another example, the physician may not be able to obtain a high-quality ultrasound image of the fetal spine by operating the ultrasound probe autonomously. This may affect the accuracy of the diagnosis.

The physician can place the ultrasound probe at any position of the fetus, for example, at a middle position of the fetus. The ultrasound imaging device can control the ultrasound probe to collect volumetric ultrasound data, can set a target plane according to the volumetric ultrasound data, and can indicate to the physician to move the ultrasound probe according to position information of the target plane. The ultrasound imaging device can display first marking information. The first marking information is used to indicate a movement direction of the ultrasound probe. The physician can move the ultrasound probe according to the first marking information.

During movement of the ultrasound probe by the physician, the ultrasound imaging device can control the ultrasound probe to collect new volumetric ultrasound data, and can set a target plane according to the new volumetric ultrasound data. New position information of the target plane can be obtained by setting the target plane in the new volumetric ultrasound data. The ultrasound imaging device can indicate that movement of the ultrasound probe is to be performed according to the new position information of the target plane. The ultrasound imaging device can display first marking information. The first marking information is used to indicate a movement direction of the ultrasound probe. The physician can move the ultrasound probe according to the first marking information. The above process can be repeated until the ultrasound probe can scan the target plane. Thus, the ultrasound imaging device can set the target plane in the volumetric ultrasound data collected each time, and indicate to the physician to move the ultrasound probe according to the position information of the target plane, so as to dynamically adjust next collection of the volumetric ultrasound data. Through dynamic adjustment, the ultrasound probe gradually moves to a pose that can scan the target plane.

When the ultrasound probe can scan the target plane, the ultrasound imaging device can display second marking information. The second marking information is used to indicate that the movement of the ultrasound probe is to be stopped. The physician can stop moving the ultrasound probe according to the second marking information. The ultrasound imaging device can control the ultrasound probe to collect two-dimensional ultrasound data of the target plane, and can perform processing (e.g., rendering) on the two-dimensional ultrasound data to obtain a two-dimensional ultrasound image of the target plane. The target plane contains a conus medullaris and no less than N vertebral bodies. N may be 5, 7, 8, or the like. The physician can diagnose the fetal spine according to the two-dimensional ultrasound image of the target plane.

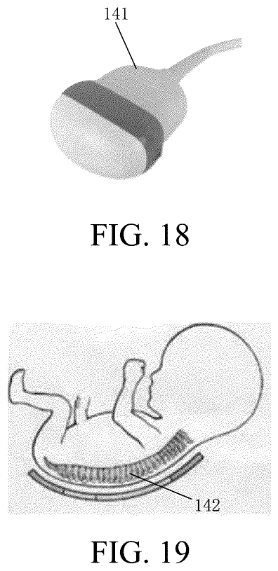

FIG. 18 shows an ultrasound probe 141. FIG. 19 shows a fetal spine 142.

Thus, by indicating that movement of the ultrasound probe is to be performed, the ultrasound imaging device can interact with the physician, thereby facilitating efficient operation of the ultrasound probe by the physician to scan the target plane. This shortens the time required for the physician to operate the ultrasound probe, and reduces the workload of the physician. In addition, the target plane contains a conus medullaris and no less than N vertebral bodies. The target plane is a preferred plane (e.g., a section) of the fetal spine. The accuracy of diagnosis can be improved by using the two-dimensional ultrasound image of the target plane.

Some embodiments of the present specification provide an ultrasound imaging method. The ultrasound imaging method can be applied to the ultrasound imaging device 100. As shown in FIG. 4, the ultrasound imaging method may include the following steps:
step 31: collecting volumetric ultrasound data by using an ultrasound probe;
step 32: setting a target plane according to the volumetric ultrasound data; and
step 33: indicating that movement of the ultrasound probe is to be performed according to position information of the target plane, so that the ultrasound probe is capable of scanning the target plane.

It should be noted that FIG. 4 shows only one possible sequence of steps, and it is not necessary to strictly follow this sequence of steps in practice. For example, some steps may be executed in parallel independently of each other. The embodiments of the present specification may include more or less steps based on conventional rules or non-inventive effort.

Thus, by indicating to the physician to move the ultrasound probe, the physician can operate the ultrasound probe targetingly. This simplifies operations for the physician, shortens the time required for the physician to operate the ultrasound probe, and reduces the workload of the physician.

In some embodiments, the target plane is a plane of a site of interest. The site of interest may be an organ or tissue, or may be a site on an organ or tissue. The site of interest has one or more target objects. The target object may be a structure or tissue on the site of interest. The target plane satisfies a set condition. The target plane can contain rich information of the site of interest. The target plane may be a preferred plane (e.g., a section) of the site of interest. When the site of interest is diagnosed based on the two-dimensional ultrasound image of the target plane, the accuracy of diagnosis can be improved.

The set condition includes a quantity condition. The set condition may include: the target plane containing a quantity of target objects greater than or equal to a set quantity. For example, the set quantity is 5, 7, 8, or the like. In this way, the target plane may contain a greater quantity of target objects. Certainly, the set condition may further include other conditions. For example, the set condition may further include a distribution condition. The set condition may further include: the target objects in the quantity greater than or equal to the set quantity being distributed along an axis of the site of interest in the target plane. In this way, the target plane may contain the axis of the site of interest. Accordingly, the target plane can contain as much information of the site of interest as possible. The target objects being distributed along the axis of the site of interest means that the target objects present a distribution trend along the axis. There is no limitation here that the target object must be located on the axis. The target object may be located on the axis or may be located in a region near the axis.

In some embodiments of step 31, the ultrasound probe can be controlled to collect volumetric ultrasound data. The volumetric ultrasound data is three-dimensional (3D) ultrasound data. Specifically, the ultrasound probe can be controlled to scan a plurality of planes within a set scanning angle range to obtain two-dimensional ultrasound data of the plurality of scanning planes. The two-dimensional ultrasound data of the plurality of scanning planes can be synthesized (e.g., interpolated) to obtain the volumetric ultrasound data. For example, each piece of two-dimensional ultrasound data can be used as a data frame; and a plurality of data frames can be synthesized by using an interpolation algorithm or other algorithms to obtain the volumetric ultrasound data. The set scanning angle range may be a maximum scanning angle range of the ultrasound probe, or may be a scanning angle range smaller than the maximum scanning angle range.

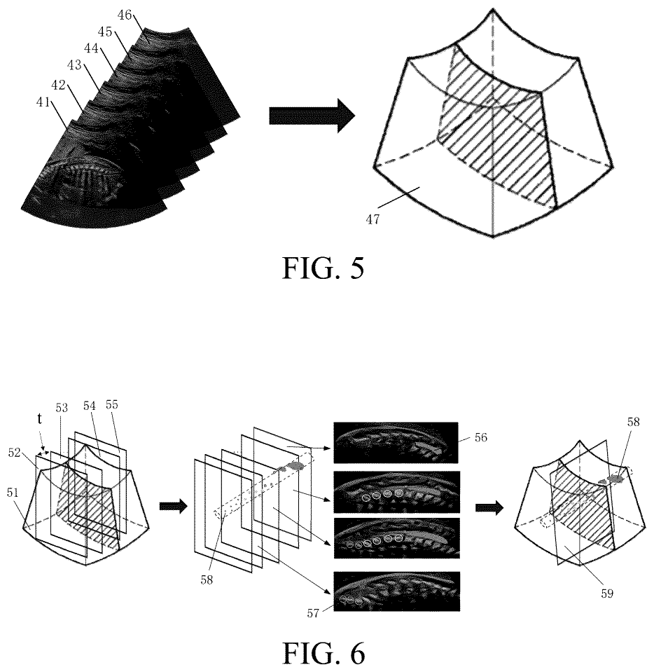

Reference is made to FIG. 5. The ultrasound probe can be controlled to collect two-dimensional ultrasound data on a first scanning plane 41, a second scanning plane 42, a third scanning plane 43, a fourth scanning plane 44, a fifth scanning plane 45 and a sixth scanning plane 46; and the two-dimensional ultrasound data of the first scanning plane 41, the two-dimensional ultrasound data of the second scanning plane 42, the two-dimensional ultrasound data of the third scanning plane 43, the two-dimensional ultrasound data of the fourth scanning plane 44, the two-dimensional ultrasound data of the fifth scanning plane 45, and the two-dimensional ultrasound data of the sixth scanning plane 46 can be synthesized to obtain volumetric ultrasound data 47.

In some embodiments, the volumetric ultrasound data can also be processed to obtain a volumetric ultrasound image. The volumetric ultrasound image can be displayed. The volumetric ultrasound image is a three-dimensional ultrasound image or a four-dimensional ultrasound image.

In some embodiments of step 32, the target plane can be set in the volumetric ultrasound data to obtain position information of the target plane. Alternatively, the target plane can be set in the volumetric ultrasound image to obtain position information of the target plane.

The position information of the target plane is used to indicate a position of the target plane in three-dimensional space. For example, the position information of the target plane is used to indicate a position of the target plane in the volumetric ultrasound data or the volumetric ultrasound image.

For example, the position information of the target plane may include position information of at least three points in the target plane, and the at least three points are non-collinear. For another example, the position information of the target plane may include position information of a point in the target plane and normal vector information of the target plane. For another example, the position information of the target plane may further include view information of the target plane.

In some embodiments of step 32, the position of the fetus in the body of the mother is not fixed but may change continuously, which results in the uncertainty of the position of the fetal spine in the body of the mother, and is not conducive to accurately setting the target plane of the fetal spine. In addition, the spine itself can also be bent toward different angles, which makes the prediction of the spine position more difficult, thereby further increasing the difficulty of setting the target plane. Thus, two-dimensional ultrasound images of a plurality of sections can be obtained according to the volumetric ultrasound data; according to the two-dimensional ultrasound image of each section, the information of the target object can be extracted from the section; and the target plane can be set according to the information of the target object in the plurality of sections. Thus, by obtaining the two-dimensional ultrasound images of the plurality of sections according to the volumetric ultrasound data, the position of the fetal spine that may appear in the body of the mother can be comprehensively covered, and the uncertainty of the fetal spine position is prevented from affecting the determination of the fetal spine position. By integrating the information of the target object in the plurality of sections, detailed information (such as position and bending direction) of the fetal spine can be comprehensively captured. In this way, the position of the fetal spine in the target and the bending direction of the fetal spine can be dynamically adapted, which is beneficial to accurately setting the target plane.

The plurality of sections can be obtained by cutting the volumetric ultrasound data or the volumetric ultrasound image along one direction. The section may be a flat surface or a curved surface. This direction may be any direction. Alternatively, the direction may be a scanning direction of the ultrasound probe (for example, an oscillation direction of the transducer element). In this way, the section may be a scanning plane of the ultrasound probe. For example, reference is made to FIG. 5. The plurality of sections may include a first scanning plane 41, a second scanning plane 42, a third scanning plane 43, a fourth scanning plane 44, a fifth scanning plane 45, and a sixth scanning plane 46.

A spacing between adjacent sections is a product of a size of the target object (e.g., a vertebral body) and a set ratio. The size of the target object includes at least one of a length and a width. The set ratio is used to enable different sections to contain the same target object, so as to set the target plane. The set ratio may be less than 1. For example, the set ratio may be ⅓, ⅔, or the like.

A target object can be identified from a two-dimensional ultrasound image to obtain information of the target object. The predetermined algorithm may be an algorithm based on an artificial intelligence (AI) model. For example, the artificial intelligence model may be a convolutional neural network (CNN) model or the like. The predetermined algorithm may further be an algorithm based on deep learning, an algorithm based on machine learning, or another algorithm.

The information of the target object is used to indicate at least one of a quantity, a position, and a size of the target object in the section. For example, the information of the target object may include at least one of quantity information, position information, and size information.

A geometric model of the site of interest can be fitted by using the predetermined algorithm according to the information of the target object in the plurality of sections. The volumetric ultrasound data or the volumetric ultrasound image can be searched according to the geometric model for a plane satisfying a set condition to serve as the target plane; and position information of the target plane can be acquired. The geometric model may be a three-dimensional model or a two-dimensional model. The geometric model is used to represent at least one of an external contour and an internal structure of the site of interest. The predetermined algorithm may be an algorithm that can perform fitting in three-dimensional space. For example, the predetermined algorithm may be an RANSAC-3D algorithm or other algorithms.

Reference is made to FIG. 6. A volumetric ultrasound image 51 can be cut at a set interval t to obtain a two-dimensional ultrasound image of a first section 52, a two-dimensional ultrasound image of a second section 53, a two-dimensional ultrasound image of a third section 54, and a two-dimensional ultrasound image of a fourth section 55. The two-dimensional ultrasound image of the first section 52, the two-dimensional ultrasound image of the second section 53, the two-dimensional ultrasound image of the third section 54, and the two-dimensional ultrasound image of the fourth section 55 can be respectively identified by using an artificial intelligence model to obtain information of a target object. The target object includes a conus medullaris 56 and a vertebral body 57. A geometric model 58 of the spine can be fitted according to the information of the target object in the first section 52, the information of the target object in the second section 53, the information of the target object in the third section 54, and the information of the target object in the fourth section 55. The volumetric ultrasound image 51 can be searched according to the geometric model 58 for a plane satisfying a set condition to serve as a target plane 59; and position information of the target plane 59 can be acquired.

As an example, the volumetric ultrasound image can be cut at a set interval to obtain two-dimensional ultrasound images of a plurality of sections; information of the target object can be extracted from the sections according to the two-dimensional ultrasound images of the sections; and a target plane can be set in the volumetric ultrasound image according to the information of the target object in the plurality of sections. The set interval may be the product of the size of the target object and the set ratio. Thus, the target plane can be set in the volumetric ultrasound image to obtain the position information of the target plane.

As another example, the volumetric ultrasound data can be segmented at a set interval to obtain two-dimensional ultrasound data of a plurality of sections; two-dimensional ultrasound images of the sections can be generated according to the two-dimensional ultrasound data of the sections; information of the target object can be extracted from the sections according to the two-dimensional ultrasound images of the sections; and a target plane can be set in the volumetric ultrasound data according to the information of the target object in the plurality of sections. The set interval may be the product of the size of the target object and the set ratio. Thus, the target plane can be set in the volumetric ultrasound data to obtain the position information of the target plane.

In some embodiments, the physician can move the ultrasound probe according to the indication of the ultrasound imaging device. During movement of the ultrasound probe by the physician, the ultrasound imaging device can control the ultrasound probe to collect new volumetric ultrasound data, and can set a target plane according to the new volumetric ultrasound data. New position information of the target plane can be obtained by setting the target plane in the new volumetric ultrasound data. The ultrasound imaging device can indicate that movement of the ultrasound probe is to be performed according to the new position information of the target plane. The above process can be repeated until the ultrasound probe can scan the target plane. Thus, the ultrasound imaging device can set a target plane in volumetric ultrasound data collected each time, and indicate to the physician to move the ultrasound probe according to position information of the target plane, so as to dynamically adjust next collection of volumetric ultrasound data. Through dynamic adjustment, the ultrasound probe gradually moves to a pose that can scan the target plane. In this way, the physician can move the ultrasound probe targetingly, which simplifies operations for the physician.

In some embodiments, the ultrasound imaging device can determine whether the ultrasound probe can scan the target plane. When the ultrasound probe can scan the target plane, the ultrasound imaging device can control the ultrasound probe to collect two-dimensional ultrasound data of the target plane, and can generate a two-dimensional ultrasound image of the target plane according to the two-dimensional ultrasound data of the target plane. When the ultrasound probe is unable to scan the target plane, the ultrasound imaging device can indicate that movement of the ultrasound probe is to be performed according to position information of the target plane.

The ultrasound probe can scan a plurality of scanning planes. As an example, whether each scanning plane satisfies a set condition can be determined respectively. When any one or a plurality of scanning planes satisfy the set condition, it can be determined that the ultrasound probe can scan the target plane. When all the scanning planes do not satisfy the set condition, it can be determined that the ultrasound probe cannot scan the target plane. For example, according to two-dimensional ultrasound data of a scanning plane, a two-dimensional ultrasound image of the scanning plane can be generated; information of the target object can be extracted from the scanning plane according to the two-dimensional ultrasound image of the scanning plane; and whether the scanning plane satisfies a set condition can be determined according to the information of the target object. For example, it can be determined whether a quantity of target objects in the scanning plane is greater than or equal to a set quantity. As another example, it can be determined whether a specific scanning plane satisfies a set condition. When the specific scanning plane satisfies the set condition, it can be determined that the ultrasound probe can scan the target plane. When the specific scanning plane does not satisfy the set condition, it can be determined that the ultrasound probe cannot scan the target plane. The specific scanning plane may be a plane where the scanning axis of the ultrasound probe is located. For example, according to two-dimensional ultrasound data of a specific scanning plane, a two-dimensional ultrasound image of the specific scanning plane can be generated; information of the target object can be extracted from the specific scanning plane according to the two-dimensional ultrasound image of the specific scanning plane; and whether the specific scanning plane satisfies a set condition can be determined according to the information of the target object.

In some embodiments of step 33, the movement may include at least one of rotation and translation. A pose of the ultrasound probe can be adjusted through movement. The pose may include at least one of a position and an attitude. The position can be adjusted by translating the ultrasound probe. The attitude can be adjusted by rotating the ultrasound probe. The rotation may include at least one of horizontal rotation and vertical rotation. Through at least one of horizontal rotation and vertical rotation, the pose of the ultrasound probe can be adjusted.

In some embodiments of step 33, the ultrasound imaging device can determine inclination information of a current scanning plane relative to the target plane according to the position information of the target plane, and can indicate that rotation of the ultrasound probe is to be performed according to the inclination information. For example, a rotation direction of the ultrasound probe can be indicated. For another example, a rotation direction and a rotation angle of the ultrasound probe can be indicated. The current scanning plane may be any scanning plane that can be scanned by the ultrasound probe. Alternatively, the current scanning plane may be a specific scanning plane. The specific scanning plane is a scanning plane where the scanning axis of the ultrasound probe is located.

The ultrasound imaging device can determine whether the ultrasound probe needs to be rotated. When the ultrasound probe needs to be rotated, an indication to rotate the ultrasound probe can be provided. When the ultrasound probe does not need to be rotated, an indication to stop rotating the ultrasound probe can be provided.

As an example, the ultrasound imaging device can calculate an inclination angle between the target plane and the current scanning plane, and can determine whether the ultrasound probe needs to be rotated according to the inclination angle. When the inclination angle is 0°, it indicates that the target plane is located on the current scanning plane, and it can be determined that the ultrasound probe does not need to be rotated. When the inclination angle is not 0°, it indicates that the target plane is not located on the current scanning plane, and it can be determined that the ultrasound probe needs to be rotated. As another example, a first similarity between the target plane and the current scanning plane can be calculated; and whether the ultrasound probe needs to be rotated can be determined according to the first similarity. When the first similarity is greater than or equal to a first threshold, it indicates that the target plane is located on the current scanning plane, and it can be determined that the ultrasound probe does not need to be rotated. When the first similarity is less than the first threshold, it indicates that the target plane is not located on the current scanning plane, and it can be determined that the ultrasound probe needs to be rotated. The first threshold can be preset. For example, two-dimensional ultrasound data of the target plane can be extracted from the volumetric ultrasound data; and a two-dimensional ultrasound image of the target plane can be generated according to the extracted two-dimensional ultrasound data. A two-dimensional ultrasound image of the current scanning plane can be generated according to the two-dimensional ultrasound data of the current scanning plane. In this way, the first similarity can be calculated according to the two-dimensional ultrasound image of the target plane and the two-dimensional ultrasound image of the current scanning plane.

Rotation information can be determined according to the inclination information; and an indication to rotate the ultrasound probe can be provided according to the rotation information.

The inclination information may include at least one of an inclination direction and an inclination angle. The inclination direction may include at least one of a horizontal inclination direction and a vertical inclination direction. The inclination angle may include at least one of a horizontal inclination angle and a vertical inclination angle. The rotation information may include a rotation direction. Alternatively, the rotation information may include a rotation direction and a rotation angle. The rotation direction may include at least one of a horizontal rotation direction and an inclined rotation direction. The rotation angle may include at least one of a horizontal rotation angle and a vertical rotation angle. For example, the horizontal rotation direction can be indicated according to at least one of the horizontal inclination direction and the horizontal inclination angle. Alternatively, the horizontal rotation direction and the horizontal rotation angle can be indicated according to the horizontal inclination direction and the horizontal inclination angle. For another example, the vertical rotation direction can be indicated according to at least one of the vertical inclination direction and the vertical inclination angle. Alternatively, the vertical rotation direction and the vertical rotation angle can be indicated according to the vertical inclination direction and the vertical inclination angle.

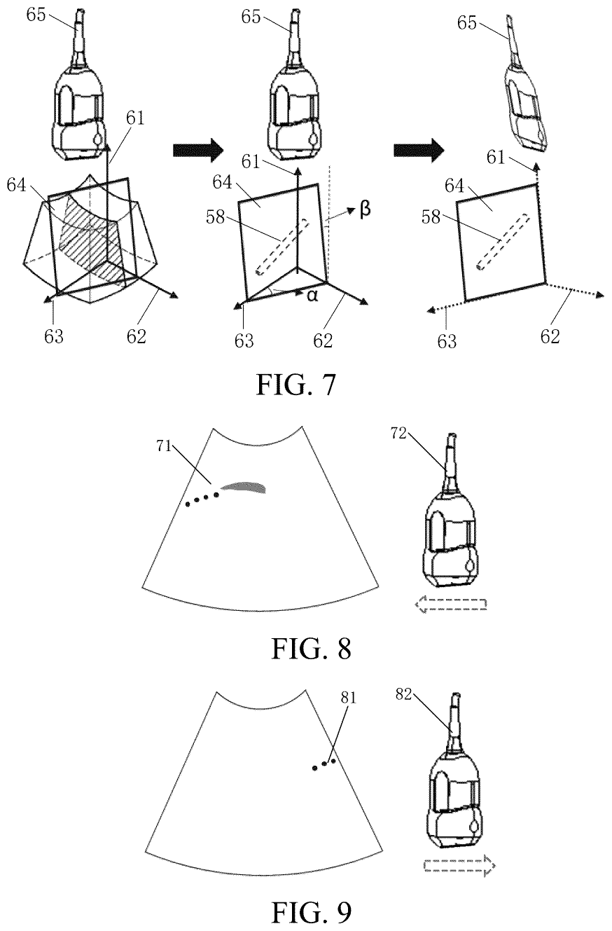

Reference is made to FIG. 7. A three-dimensional rectangular coordinate system can be established with a direction of the scanning axis (a vertical direction) as a direction of a Z axis 61 and a scanning direction (e.g., an oscillation direction of the transducer element) as a direction of an X axis 62. The three-dimensional rectangular coordinate system satisfies the left-hand rule or the right-hand rule. The current scanning plane is located on a plane formed by the Z axis 61 and a Y axis 63.

Coordinate information of the target plane in the three-dimensional rectangular coordinate system can be obtained according to the information of the target plane 64; and a horizontal inclination angle α and a vertical inclination angle β of the current scanning plane relative to the target plane 64 can be calculated according to the coordinate information. The horizontal rotation direction can be determined according to the horizontal inclination angle α. The horizontal rotation direction points to a direction where α decreases. The vertical rotation direction can be determined according to the vertical inclination angle β. The vertical rotation direction points to a direction where β decreases. Rotating the ultrasound probe 65 along the horizontal rotation direction and rotating the ultrasound probe 65 along the vertical rotation direction can cause the target plane 64 to be located on the current scanning plane.

The rotation direction can point to a direction where the inclination angle decreases. By rotating the ultrasound probe along the rotation direction, the target plane can be located on the current scanning plane. When the target plane is located on the current scanning plane, an indication to stop rotating the ultrasound probe can be provided. The target plane being located on the current scanning plane may mean that the current scanning plane can cover the entire target plane. Thus, the ultrasound probe can scan the target plane, and there is no need to provide an indication to translate the ultrasound probe. Alternatively, the target plane is located on the current scanning plane, or the current scanning plane can cover a part of the target plane. Thus, the ultrasound probe cannot scan the target plane. An indication to translate the ultrasound probe can be provided to enable the current scanning plane to cover the entire target plane.

The physician can rotate the ultrasound probe according to the indication of the ultrasound imaging device. During rotation of the ultrasound probe, the ultrasound imaging device can determine whether it is necessary to continue rotating the ultrasound probe. When the ultrasound probe needs to be rotated, the ultrasound imaging device can indicate that rotation of the ultrasound probe is to be performed. When the ultrasound probe does not need to be rotated, the ultrasound imaging device can indicate that the rotation of the ultrasound probe is to be stopped.

In some embodiments of step 33, the ultrasound imaging device can extract information of the target object from the current scanning plane according to the two-dimensional ultrasound image of the current scanning plane, and can indicate that translation of the ultrasound probe is to be performed according to the information of the target object. The current scanning plane may be any scanning plane that can be scanned by the ultrasound probe. Alternatively, the current scanning plane may be a specific scanning plane. The specific scanning plane is a scanning plane where the scanning axis of the ultrasound probe is located.

The ultrasound imaging device can determine whether the ultrasound probe needs to be translated. When the ultrasound probe needs to be translated, an indication to translate the ultrasound probe can be provided. When the ultrasound probe does not need to be translated, an indication to stop translating the ultrasound probe can be provided.

As an example, the ultrasound imaging device can calculate a second similarity between the two-dimensional ultrasound image of the current scanning plane and a reference ultrasound image, and can determine whether the ultrasound probe needs to be translated according to the second similarity. When the second similarity is greater than or equal to a second threshold, it indicates that the current scanning plane can cover the entire target plane, and it can be determined that the ultrasound probe does not need to be translated. When the second similarity is less than the second threshold, it indicates that the current scanning plane cannot cover the entire target plane, and it can be determined that the ultrasound probe needs to be translated. The second threshold can be preset. The reference ultrasound image may be a preset two-dimensional ultrasound image. The reference ultrasound image can satisfy a set condition. As another example, quantity information of the target object can be extracted from the current scanning plane according to the two-dimensional ultrasound image of the current scanning plane. Whether the ultrasound probe needs to be translated can be determined according to the quantity information of the target object. When a quantity of target objects in the current scanning plane is greater than or equal to a set quantity, it indicates that the ultrasound probe can scan the target plane, and it can be determined that the ultrasound probe does not need to be translated. When the quantity of the target objects in the current scanning plane is less than the set quantity, it indicates that the ultrasound probe cannot scan the target plane, and it can be determined that the ultrasound probe needs to be translated.

Position information of the target object can be extracted from the current scanning plane according to the two-dimensional ultrasound image of the current scanning plane. The position information of the target object is used to indicate a position of the target object in the current scanning plane. A translation direction can be indicated according to the position information of the target object. For example, distances between the target plane and boundaries of the current scanning plane in a plurality of directions can be calculated according to the position information of the target object; and a direction with the minimum distance can be selected as the translation direction of the ultrasound probe. For example, the plurality of directions above may include four directions. The four directions may include two opposite directions along one straight line and two opposite directions along another straight line. The two straight lines are perpendicular to each other.

Reference is made to FIG. 8. The target object 71 is located on a left side of the current scanning plane. The translation direction of the ultrasound probe 72 is to the left. Reference is made to FIG. 9. The target object 81 is located on a right side of the current scanning plane. The translation direction of the ultrasound probe 82 is to the right.

The physician can translate the ultrasound probe according to the indication of the ultrasound imaging device. During translation of the ultrasound probe, the ultrasound imaging device can determine whether it is necessary to continue translating the ultrasound probe. When the ultrasound probe needs to be translated, the ultrasound imaging device can indicate that translation of the ultrasound probe is to be performed. When the ultrasound probe does not need to be translated, the ultrasound imaging device can indicate that the translation of the ultrasound probe is to be stopped.

In some embodiments of step 33, a priority of rotation can be higher than a priority of translation. First, it can be determined whether the ultrasound probe needs to be rotated. When the ultrasound probe needs to be rotated, an indication to rotate the ultrasound probe can be provided. When the ultrasound probe does not need to be rotated, an indication to stop rotating the ultrasound probe can be provided. Then, it can be determined whether the ultrasound probe needs to be translated. When the ultrasound probe needs to be translated, an indication to translate the ultrasound probe can be provided. When the ultrasound probe does not need to be translated, an indication to stop translating the ultrasound probe can be provided.

Thus, an indication to rotate the ultrasound probe can be provided to the physician first, enabling the target plane to be located on the current scanning plane. After the target plane is located on the current scanning plane, an indication to translate the ultrasound probe is provided to the physician, enabling the current scanning plane to cover the entire target plane. When the current scanning plane can cover the entire target plane, the ultrasound probe can scan the target plane.

In some embodiments of step 33, when the ultrasound probe needs to be moved, corresponding first marking information can be acquired according to the movement direction of the ultrasound probe; and the first marking information can be displayed. The first marking information is used to indicate a movement direction of the ultrasound probe. The first marking information may include a text, a pattern, a symbol, or the like. For example, the first marking information may be an arrow. A direction of the arrow is the movement direction. Thus, the indication to move the ultrasound probe can be provided in a visualized form.

The first marking information may include at least one of first sub-marking information and second sub-marking information. The first sub-marking information is used to indicate a rotation direction of the ultrasound probe. The corresponding first sub-marking information can be acquired according to the rotation direction of the ultrasound probe; and the first sub-marking information can be displayed. The second sub-marking information is used to indicate a translation direction of the ultrasound probe. The corresponding second sub-marking information can be acquired according to the translation direction of the ultrasound probe; and the second sub-marking information can be displayed.

Certainly, the indication to move the ultrasound probe can be provided in other forms. For example, the indication to move the ultrasound probe can be provided in the form of a sound. The sound can indicate the movement direction of the ultrasound probe. The sound may be a speech or a prompt tone.

In some embodiments, during movement of the ultrasound probe by the physician, the ultrasound imaging device can control the ultrasound probe to collect volumetric ultrasound data in real time, can set a target plane according to the volumetric ultrasound data, and can indicate that movement of the ultrasound probe is to be performed according to position information of the target plane. Thus, interaction between the ultrasound imaging device and the physician is achieved. Through this interaction, the physician can continuously move the ultrasound probe until the ultrasound probe can scan the target plane.

Optionally, the ultrasound imaging device can control the ultrasound probe to scan a plurality of planes within a set scanning angle range to obtain two-dimensional ultrasound data of the plurality of scanning planes, and can synthesize the two-dimensional ultrasound data of the plurality of scanning planes to obtain volumetric ultrasound data. During movement of the ultrasound probe by the physician, the ultrasound imaging device can adjust the scanning angle range of the ultrasound probe. The scanning angle range is anti-correlated with the degree of orientation deviation between the current scanning plane and the target plane. Thus, during movement of the ultrasound probe by the physician, as the current scanning plane continuously approaches the target plane, the scanning angle range can be continuously reduced, thereby reducing the size of the volumetric ultrasound data and the amount of calculation, shortening the time for scanning the site of interest, and improving the efficiency.

The degree of orientation deviation can be measured by the inclination information. For example, the degree of orientation deviation can be measured by the inclination angle between the current scanning plane and the target plane. Thus, the ultrasound imaging device can adjust the scanning angle range according to the inclination information. The ultrasound imaging device can calculate the adjusted scanning angle range by using a rule, a mathematical formula, or other methods.

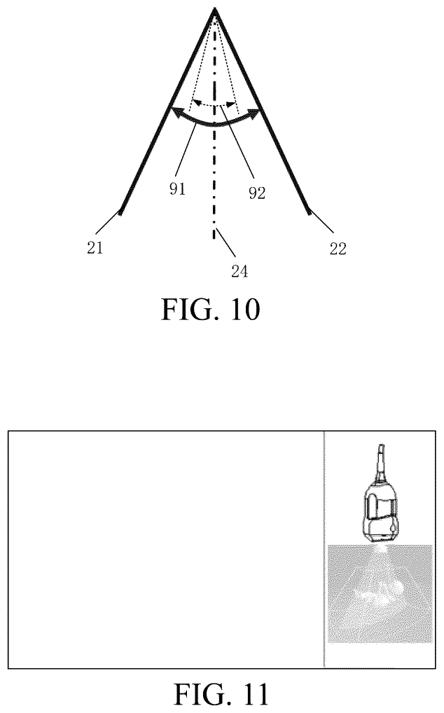

For example, in FIGS. 10, 91 denotes a scanning angle range before adjustment, and 92 denotes a scanning angle range after adjustment.

In some embodiments, during movement of the ultrasound probe by the physician, the ultrasound imaging device can acquire a two-dimensional ultrasound image of the current scanning plane, and display the two-dimensional ultrasound image of the current scanning plane. Thus, during movement of the ultrasound probe by the physician, displaying the two-dimensional ultrasound image of the current scanning plane in real time by the ultrasound imaging device is convenient for the physician to intuitively understand the current scanning plane of the ultrasound probe. The ultrasound probe can scan a plurality of scanning planes. The current scanning plane may be any one of the plurality of scanning planes, or may be a specific scanning plane among the plurality of scanning planes.

In some embodiments, second marking information can be acquired when the ultrasound probe does not need to be moved; and the second marking information can be displayed. The second marking information is used to indicate that the movement of the ultrasound probe is to be stopped. The second marking information may include a text, a pattern, a symbol, or the like. Thus, the indication to stop moving the ultrasound probe can be provided in a visualized form.

The second marking information may include at least one of third sub-marking information and fourth sub-marking information. The third sub-marking information is used to indicate that the rotation of the ultrasound probe is to be stopped. The fourth sub-marking information is used to indicate that the translation of the ultrasound probe is to be stopped.

Certainly, the indication to move the ultrasound probe can be provided in other forms. For example, the indication to stop moving the ultrasound probe can be provided in the form of a sound. The sound may be a speech, a prompt tone, or the like.

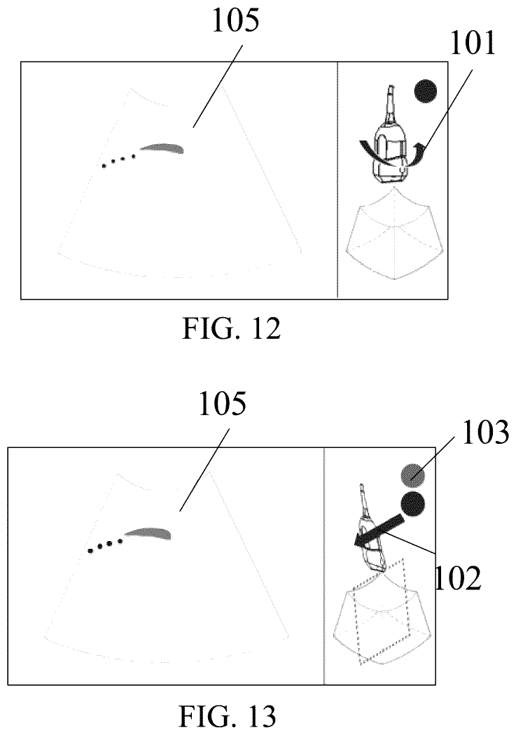

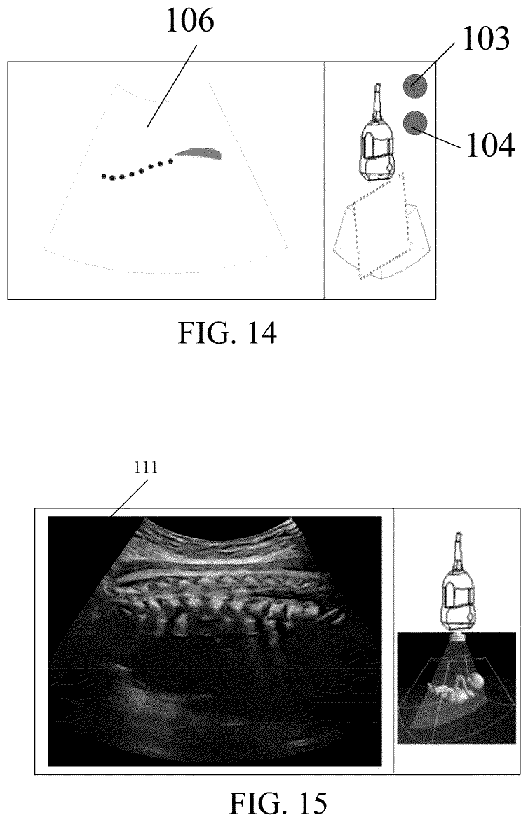

For example, FIG. 11, FIG. 12, FIG. 13, and FIG. 14 each show an interaction interface. In FIG. 11, the ultrasound imaging device collects volumetric ultrasound data. In FIG. 12, the first sub-marking information 101 is used to indicate to the physician to rotate the ultrasound probe. In FIG. 13, the second sub-marking information 102 is used to indicate to the physician to translate the ultrasound probe. The third sub-marking information 103 is used to indicate to the physician to stop rotating the ultrasound probe. In FIG. 14, the third sub-marking information 103 is used to indicate to the physician to stop rotating the ultrasound probe. The fourth sub-marking information 104 is used to indicate to the physician to stop translating the ultrasound probe. In the interaction interfaces of FIG. 12 and FIG. 13, a two-dimensional ultrasound image 105 of the current scanning plane is displayed. In the interaction interface of FIG. 14, a two-dimensional ultrasound image 106 of the target plane is displayed.

In some embodiments, the ultrasound probe may be a mechanical scanning ultrasound probe. The ultrasound probe includes a movable transducer element. When the ultrasound probe can scan the target plane, the ultrasound imaging device can control the transducer element to stop moving. Thus, the ultrasound imaging device can operate in a two-dimensional scan mode, which facilitates collection of the two-dimensional ultrasound data of the target plane.

In some embodiments, when the ultrasound probe can scan the target plane, the ultrasound imaging device can collect two-dimensional ultrasound data of the target plane by using the ultrasound probe, and generate a two-dimensional ultrasound image according to the two-dimensional ultrasound data of the target plane.

For example, FIG. 15 shows an interaction interface. The interaction interface includes a two-dimensional ultrasound image 111 of the target plane.

In some embodiments, the physician can make a diagnosis according to the two-dimensional ultrasound image of the target plane. Alternatively, the ultrasound imaging device can make a diagnosis according to the two-dimensional ultrasound image of the target plane to obtain a diagnosis result. For example, the ultrasound imaging device can diagnose the two-dimensional ultrasound image based on an artificial intelligence model to obtain a diagnosis result. The diagnosis result is used to indicate a health condition of the site of interest. Thus, the ultrasound imaging device can automatically give the diagnosis result, which saves the workload of the physician.

A specific scenario example according to an embodiment of the present specification will be described below.

The ultrasound imaging device can collect volumetric ultrasound data, and perform processing on the volumetric ultrasound data to obtain a volumetric ultrasound image. The ultrasound imaging device can determine whether the ultrasound probe can scan the target plane according to the volumetric ultrasound image. When the ultrasound probe can scan the target plane, the ultrasound imaging device can control the ultrasound probe to collect two-dimensional ultrasound data of the target plane, and can generate a two-dimensional ultrasound image of the target plane according to the two-dimensional ultrasound data of the target plane. When the ultrasound probe is unable to scan the target plane, the ultrasound imaging device can set a target plane in the volumetric ultrasound image, and can indicate that movement of the ultrasound probe is to be performed according to position information of the target plane. The physician can move the ultrasound probe according to the indication of the ultrasound imaging device. During movement of the ultrasound probe by the physician, the ultrasound imaging device can control the ultrasound probe to collect new volumetric ultrasound data, and can perform processing on the new volumetric ultrasound data to obtain a new volumetric ultrasound image. The ultrasound imaging device can determine whether the ultrasound probe can scan the target plane according to the new volumetric ultrasound image. When the ultrasound probe can scan the target plane, the ultrasound imaging device can control the ultrasound probe to collect two-dimensional ultrasound data of the target plane, and can generate a two-dimensional ultrasound image of the target plane according to the two-dimensional ultrasound data of the target plane. When the ultrasound probe is unable to scan the target plane, the ultrasound imaging device can set a target plane in the new volumetric ultrasound image, and can indicate that movement of the ultrasound probe is to be performed according to new position information of the target plane.

Thus, the two-dimensional ultrasound image of the target plane can be obtained by dynamically adjusting the position of the ultrasound probe.

In the technical solutions of the embodiments of the present specification, an ultrasound probe may be used to collect volumetric ultrasound data. The target plane can be set in the volumetric ultrasound data. An indication to move the ultrasound probe can be provided according to position information of the target plane, so that the ultrasound probe is capable of scanning the target plane. Thus, by indicating to the physician to move the ultrasound probe, the physician can operate the ultrasound probe targetingly. This simplifies operations for the physician, shortens the time required for the physician to operate the ultrasound probe, and reduces the workload of the physician. In addition, the target plane is the plane of the fetal spine that satisfies the set condition. The ultrasound probe can scan the target plane of the fetal spine to obtain a two-dimensional ultrasound image of the target plane, which is convenient for the physician to diagnose the fetal spine.

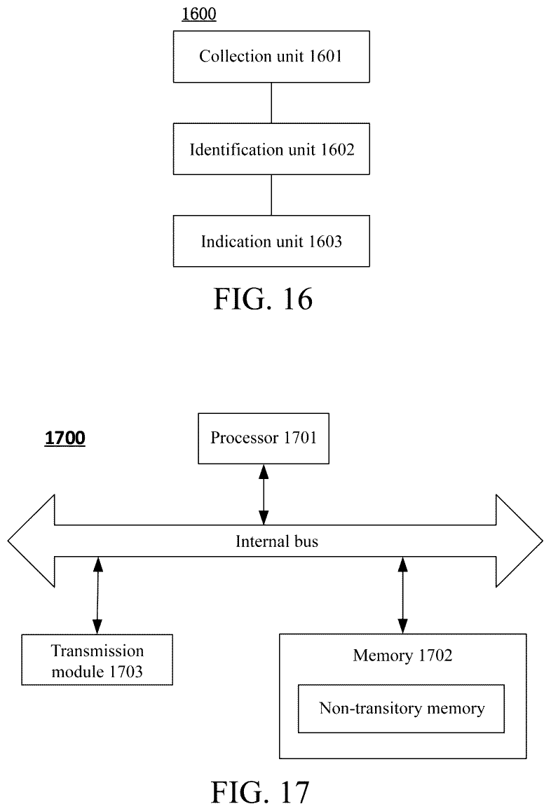

As shown in FIG. 16, some embodiments of the present specification further provide an ultrasound imaging apparatus. The ultrasound imaging apparatus is applicable to the ultrasound imaging device 100. As shown in FIG. 16, the ultrasound imaging apparatus 1600 may include the following units:
a collection unit 1601, configured to collect volumetric ultrasound data by using an ultrasound probe;
a setting unit 1602, configured to set a target plane according to the volumetric ultrasound data; and
an indication unit 1603, configured to indicate that movement of the ultrasound probe is to be performed according to position information of the target plane, so that the ultrasound probe is capable of scanning the target plane, wherein the target plane is a plane of a spine that satisfies a set condition.

It should be noted that since the implementation solution for problem-solving by the above apparatus is similar to that of the above method, for the specific implementation of the apparatus in the embodiments of the present specification, reference can be made to the implementation of the above method, and repeated details are omitted. The term “unit” used below may be a combination of software and/or hardware that implements a predetermined function. Although the apparatus described in the following embodiments is preferably implemented by software, implementation by hardware or a combination of software and hardware is also possible and conceived of.

The embodiments of the present specification further provide an ultrasound imaging apparatus. FIG. 17 is a schematic architecture diagram of an ultrasound imaging apparatus. As shown in FIG. 17, the ultrasound imaging apparatus 1700 may include a processor 1701 and a memory 1702. The memory 1702 may include a non-transitory memory. The non-transitory memory, also referred to as a non-volatile memory, stores information that will not be lost after power is turned off. The non-transitory memory may include a magnetic storage apparatus, a flash memory, an optical disk, and the like. The memory 1702 may further include a volatile memory such as a high-speed random access memory. The memory 1702 is configured to store instructions. The instructions, when executed, enable the processor 1701 to execute the ultrasound imaging method according to the foregoing embodiment.

The ultrasound imaging apparatus 1700 may further include a transmission module 1703 for a communication function. The transmission module 1703 is configured to receive or send data via a network. The specific examples of the network described above may include a wireless network provided by a communication supplier of the computer terminal. In an example, the transmission module 1703 includes a network interface controller (NIC) which may be connected to other network devices by means of a base station to communicate with the Internet. In an example, the transmission module 1703 may be a radio frequency (RF) module which is configured to communicate with the Internet in a wireless manner.

It can be understood by those of ordinary skill in the art that the structure shown in FIG. 17 is only illustrative and does not limit the structure of the ultrasound imaging apparatus. For example, the ultrasound imaging apparatus may further include more or fewer assemblies than shown in FIG. 17.

The embodiments of the present specification further provide a non-transitory computer-readable storage medium for storing a computer program, where the computer program, when executed by a computer, enables the computer to execute the ultrasound imaging method according to the foregoing embodiment.

The non-transitory computer-readable storage medium may include: apparatuses that store information using electrical energy, such as various memories, e.g., RAM, ROM, etc., apparatuses that store information using magnetic energy, such as hard disks, floppy disks, magnetic tapes, magnetic core memories, bubble memories, and U disks; and apparatuses that store information using optical means, such as CDs or DVDs. Certainly, there are other forms of readable storage media, such as a quantum memory, a graphene memory, etc.

The embodiments of the present specification further provide a computer program product, including a computer program. The computer program, when executed by a processor, implements the ultrasound imaging method according to the foregoing embodiment.

It can be understood by those skilled in the art that the present specification may be provided as a method, a system, or a computer program product. Therefore, the present specification may be implemented in the form of a fully hardware-based embodiment, a fully software-based embodiment, or an embodiment combining software and hardware. Furthermore, the present specification may be implemented in the form of a computer program product implemented on one or more computer-usable storage media (including, but not limited to, magnetic disk storage, CD-ROM, optical storage, etc.) having computer-usable program codes included therein.

The present specification is described with reference to flowcharts and/or block diagrams of methods, devices (systems), and computer program products according to the embodiments of the present specification. It should be understood that each flow and/or block in the flowcharts and/or block diagrams and a combination of flows and/or blocks in the flowcharts and/or block diagrams may be implemented by computer program instructions. The computer may be a personal computer, a laptop computer, a cellular telephone, a camera phone, a smart phone, a personal digital assistant, a media player, a navigation device, an email device, a gaming console, a tablet computer, a wearable device, or a combination of any of these devices.

Each functional unit in the embodiments of the present specification may be integrated into one processing unit, or each functional unit may exist alone physically, or two or more functional units may be integrated into one processing unit.

It can be understood by those skilled in the art that the description of each embodiment in the present specification has its own focus, and for a part not described in detail in a certain embodiment, reference may be made to the related description of other embodiments. In addition, it may be understood that any combination of some or all of the embodiments described in the present specification can be conceived by those skilled in the art without the exercise of any inventive effort after reading the document of the present specification, and such combinations are also within the scope of the disclosure and protection of the present specification.

Although the present specification has been described by means of embodiments, those of ordinary skill in the art will appreciate that the above embodiments are only used to facilitate understanding of the core idea of the present specification. It can be understood by those skilled in the art that many modifications and variations of the present specification are possible. It is intended that the appended claims cover such modifications and variations without departing from the spirit of the present specification.

1. An ultrasound imaging method, comprising:
obtain volumetric ultrasound data;
setting a target plane according to the volumetric ultrasound data; and
indicating that movement of the ultrasound probe is to be performed according to position information of the target plane, so that the ultrasound probe is capable of scanning the target plane, wherein the target plane is a plane of a fetal spine that satisfies a set condition.

2. The method according to claim 1, wherein
the set condition comprises the target plane containing a quantity of target objects greater than or equal to a set quantity; and
the target object comprises at least one of a conus medullaris and a vertebral body.

3. The method according to claim 2, wherein
the setting a target plane according to the volumetric ultrasound data comprises:
acquiring two-dimensional ultrasound images of a plurality of sections according to the volumetric ultrasound data;
extracting information of the target object in the plurality of sections according to the two-dimensional ultrasound images of the plurality of sections; and
setting the target plane according to the information of the target object in the plurality of sections.

4. The method according to claim 3, wherein
a spacing between adjacent sections is a product of a size of the target object and a set ratio.

5. The method according to claim 2, wherein the movement comprises at least one of rotation and translation.

6. The method according to claim 5, wherein
the indicating that movement of the ultrasound probe is to be performed comprises:
determining inclination information of a current scanning plane relative to the target plane according to the position information of the target plane; and
indicating that rotation of the ultrasound probe is to be performed according to the inclination information.

7. The method according to claim 5, wherein
the indicating that movement of the ultrasound probe is to be performed comprises:
extracting information of the target object from a current scanning plane according to a two-dimensional ultrasound image of the current scanning plane; and
indicating that translation of the ultrasound probe is to be performed according to the information of the target object.

8. The method according to claim 1, wherein
the indicating that movement of the ultrasound probe is to be performed comprises:
displaying first marking information, the first marking information being used to indicate a movement direction of the ultrasound probe.

9. The method according to claim 1, further comprising:
displaying second marking information, the second marking information being used to indicate that the movement of the ultrasound probe is to be stopped.

10. The method according to claim 1, further comprising:
collecting two-dimensional ultrasound data of the target plane by using the ultrasound probe; and
generating a two-dimensional ultrasound image according to the two-dimensional ultrasound data of the target plane.

11. The method according to claim 1, wherein
a scanning angle range of the ultrasound probe is anti-correlated with a degree of orientation deviation between a current scanning plane and the target plane.

12. The method according to claim 1, wherein the ultrasound probe comprises a movable transducer element; and
the method further comprises:
controlling the transducer element to stop moving when the ultrasound probe is capable of scanning the target plane.

13. An ultrasound imaging device, comprising:
an ultrasound probe, configured to collect volumetric ultrasound data;
a memory storing instructions; and
a processor configured to execute the instructions to:
set a target plane according to the volumetric ultrasound data; and
indicate that movement of the ultrasound probe is to be performed according to position information of the target plane, so that the ultrasound probe is capable of scanning the target plane, wherein the target plane is a plane of a fetal spine that satisfies a set condition.

14. A non-transitory computer-readable storage medium for storing a computer program that, when executed by a computer, causes the computer to:
obtain volumetric ultrasound data;
set a target plane according to the volumetric ultrasound data; and
indicate that movement of the ultrasound probe is to be performed according to position information of the target plane, so that the ultrasound probe is capable of scanning the target plane, wherein the target plane is a plane of a fetal spine that satisfies a set condition.
